(19) United States
(12) Patent Application Publication
(10) Pub. No.: US 2018/0271753 A1
(43) Pub. Date: Sep. 27, 2018

(54) MEDICATION DISPENSING PHONE CASE SYSTEM

(71) Applicant: HealthBeacon Ltd., Dublin (IE)

(72) Inventors: James Joyce, Needham, MA (US); Kieran Daly, Dublin (IE)

(73) Assignee: HealthBeacon Ltd., Dublin (IE)

(21) Appl. No.: 15/934,022

(22) Filed: Mar. 23, 2018

Related U.S. Application Data
(60) Provisional application No. 62/475,370, filed on Mar. 23, 2017.

(51) Int. Cl. A61J 7/00; A61J 7/04; G16H 20/13; G06K 7/10; G16H 15/00

(52) U.S. Cl. CPC A61J 7/0076; A61J 7/0409; G16H 20/13; G06K 7/10366; H04W 4/80; A61J 2200/30; A61J 2200/72; A61J 2200/74; A61J 2205/60; G16H 15/00

(57) ABSTRACT

An apparatus and method for a medication dispensing phone case system. A system includes a smartphone, the smartphone including at least a processor, a memory and a display, the memory including at least an operating system and an adherence app, a connector system, the connector system removably linking the smartphone to a medication dispensing container, the connector system including at least sensing technology that enables the connector system to read an activity from the medication dispensing container, and a server in a network of interconnected computer devices, the server communicatively linked to the smartphone to enable interaction between the adherence app and an adherence process residing in the server.

### CROSS REFERENCE TO RELATED APPLICATIONS

This application claims benefit from U.S. Provisional Patent Application Ser. No. 62/475,370, filed Mar. 23, 2017, which is incorporated by reference in its entirety.

### STATEMENT REGARDING GOVERNMENT INTEREST

None.

### BACKGROUND OF THE INVENTION

The present invention generally relates to medical adherence, and more specifically to a medication dispensing phone case system.

In general, medication adherence usually refers to whether patients take their medications as prescribed (e.g., twice daily), as well as whether they continue to take a prescribed medication. Medication non-adherence is a growing concern to clinicians, healthcare systems, and other stakeholders (e.g., payers) because of mounting evidence that it is prevalent and associated with adverse outcomes and higher costs of health care. To date, measurement of patient medication adherence and use of interventions to improve adherence are rare in routine clinical practice.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention relates generally to medical adherence, and more specifically to a medication dispensing phone case system.

In one aspect, the invention features a system including a smartphone linked to a dispensing unit by a connector system, the smartphone enabled to detect one or more items dispensed from the dispensing unit, the smartphone in communication with a content delivery network (CDN) including at least an adherence server.

In another aspect, the invention features a system including a smartphone, the smartphone including at least a processor, a memory and a display, the memory including at least an operating system and an adherence app, a connector system, the connector system removably linking the smartphone to a medication dispensing container, the connector system including at least sensing technology that enables the connector system to read an activity from the medication dispensing container, and a server in a network of interconnected computer devices, the server communicatively linked to the smartphone to enable interaction between the adherence app and an adherence process residing in the server.

In still another aspect, the invention features a method including enclosing a smartphone with a connector system, linking the connector system to a dispensing unit, and sensing an activity associated with the dispensing unit.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is an illustration of a first embodiment of an exemplary medication adherence system.

FIG. 2 is an illustration of a second embodiment of an exemplary medication adherence system.

FIG. 3 is an illustration of a third embodiment of an exemplary medication adherence system.

FIG. 4 is a flow diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

In medicine, compliance (also adherence, capacitance) describes the degree to which a patient correctly follows medical advice. Most commonly, it refers to medication or drug compliance, but it can also apply to other situations such as medical device use, self care, self-directed exercises, or therapy sessions. Compliance, adherence, and persistence are all terms commonly used to describe medication-taking behaviors. Adherence to, or compliance with, a medication regimen is generally defined as the extent to which a person takes medications as prescribed by their healthcare providers. Taking medication correctly may seem like a simple or personal matter, but non-adherence (or not taking medication as directed) is a complicated and common problem. People do not realize the real damage or consequences of non-adherence. When patients with chronic conditions do not take medication as directed, the repercussions can be severe.

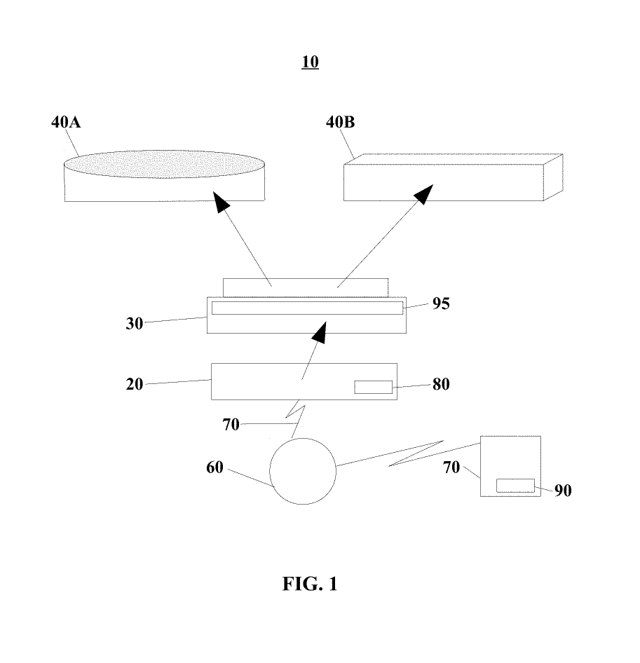

As shown in FIG. 1, a first embodiment of an exemplary medication adherence system 10 includes a smartphone 20, a phone case connector system 30 and a dispensing unit 40. In the embodiment shown, the dispensing unit 40 may be a cream dispensing unit 40A or a pill dispensing unit 40B. Although only two alternate dispensing units 40A, 40B, are illustrated, other types of dispensing units may be adapted to the medication adherence system 10. The smartphone 20 is linked to a wireless network 50 that enables the smartphone 20 to communicate with a content delivery network or content distribution network (CDN) that is a globally distributed network of servers 60 deployed in multiple data centers. One the of the servers 60 residing in the CDN and accessible from the smartphone is an adherence server 70.

The smartphone 20 can include, for example, an operating system (O/S), such as Android® or IOS®, a processor, a memory and a display. The memory can include one or more application programs, generally referred to as “apps.” One such app resident in the memory of the smartphone 20 is a managing medicine adherence app 80, which, for example, tracks adherence, expiry of medication, interacts with clinical support, re-orders medications, and so forth. The managing medicine adherence app 80 can also remind a smartphone user (e.g., patient) when the user needs to take one or more of their medications and let the patient know if they have forgotten to take their medications. The reminder, a signal generated by the smartphone 20, can include one or more of an alarm, a light, a sound, and so forth. The managing medicine adherence app 80 can provide the patient with a history of when they have taken their medications. The managing medicine adherence app 80 can show the patient a patient adherence score, how much of their current supply of medication is left, have an ability to reorder medication and determine if and when medication is taken. The managing medicine adherence app 80 also enables communication with the adherence server 70, caregivers and family members. For example, a caregiver or family member, in response to a text message from the patient's smartphone 20, can communicate directly with the patient through voice, email or return text message.

The medical adherence server 70 can include a processor and memory. The memory can include at least an operating system, such as Windows or Linux, and a medication adherence process 90.

The smartphone 20 is enclosed in the phone case connector system 30. In one embodiment, the connector system 30 includes a smartphone case or jacket. The connector system 30 is linked to medical dispensing unit 40, such as the cream dispensing unit 40A, the pill dispensing unit 40B, and so forth. The link may be indirect, such as a clasp, or direct, such as integral to both the connector system 30 and medication dispensing unit 40. The connector system 30 includes sensing technology 95 that enables the connector system 30 to read an activity from any attached medication dispensing container 40. Sensing technology 95 may include, for example, Bluetooth®, Near Field Communication (NFC), Radio Frequency Identification (RFID) and so forth. In some implementations, the connector system 30 may also connect directly to an injectable unit, an infusion pump, a sharps container, and so forth.

The sensed or detected activity may be a movement, a temperature change, an air pressure change, a weight change, and so forth. The movement is a content movement occurring between the dispensing system 40 and the connector system 30, the contents or some portion thereof. One objective of the connector system 30 is to observe and record patient behavior by the sensed or detected activity to determine if, for example, the patient has taken the medication, is the medication in temperature, and in date (not expired). This information can be made available to, for example, a care team (i.e., nurse), a prescribing clinician, and a pharmacist to support the patient when they don't comply to a schedule or require new product.

The medication dispensing phone case system 10 when companioned with both the smartphone 20 and the smartphone app 80, can collect, process, integrate, and analyze environmental information and combine it with patient sensing data and patient profiles. This information and analysis can then drive patient intervention and patient communication with their clinicians (e.g., doctors, nurses and pharmacists) and care givers. The medication phone case dispensing system 10 integrates with the app 80 on the patient phone 20, which enables the patient to communicate through the system 10. In one example the system 10 acts as an intelligent agent, commonly referred to as artificially intelligent. The system 10 is able to perceive the environment, learn and problem solve, and then take actions by communicating to the patient through the system 10 by triggering a communication by voice, email, SMS, alarm(s), sound(s), visual alert(s), color change of device, vibration and other communication and alert that increases and maximizes the chance that the patient takes the medication on time and/or interacts with the system 10. The system 10 sends similar communications to the clinicians and/or caregivers that drive and suggest targeted patient interventions. The system 10 enables the dispensing unit 40 to mimic cognitive and communication functions that are generally associated with human intelligence and human interactions. In one example, the system 10 understands human speech and interprets complex data and communicates directly with the patient. The clinicians are able to directly adjust treatments and then the system 10 communicates those treatments directly to the patient. The system 10 is able to ask the patient basic health questions, observe their speech, observe vital health signals from other applications and connected devices including, temperature, heart rate, steps, speech, sleep, and weight. The system 10 can then speak with and nudge the patient towards medical adherence and healthier outcomes. In one example, if the system 10 is unable to determine if the patient has taken the medication it can ask a confirming question.

The system 10 can remind the patient of their schedules, medication collections, deliveries, appointments, expiration, prescription renewals, medical updates and report adverse events. Moreover, the system 10 can collect disease activity markers to assess the progression of a disease or effect of the medication treatment and correlate it to medication adherence patterns. Examples of this are a patient may answer a question verbally, fill out a survey, take photo, use the accelerometer in the phone, plug in a medical device, upload clinical results, take a measurement, connect to another sensing device (scale, phone, medical equipment). This data can be reported to care teams and the data analyzed enabling medication treatment and schedules to be recommended for adjustment. The combination of medication adherence tracking and other collected sensing data that is relevant to the disease and treatment can build a database that is capable of making recommendations to improve clinical outcomes.

The system 10 enables a doctor to go online, adjust a treatment schedule that alerts and updates the patient and dispensing pharmacist. In one example, the system 10 verifies the voice of the patient before allowing the dispensing unit 40 to open through a locking or latch mechanism. In one example, the patient speaks to the system 10, the system 10 verifies the patient, updates relevant medical and tracking information, including, for example, changes in medication schedule, self reporting events, and change in patient profile information.

The exemplary medication adherence system 10 also includes a medication management companion induction charger (not shown). The charging, managing and monitoring of connected medications is a critical aspect of ensuring that patients have a functioning medication adherence system 10. The medication management companion induction charger not only charges the smartphone within the connector system, but can additionally monitor other vital patient behaviors and connectivity. The medication management companion induction charger can test and provide other quality control measures to ensure the connections among the smartphone 20, the connector system 30 and the dispensing unit 40 are valid and functioning. This monitoring can include weight, how many medicine units are on charger, temperature, use of charger, location of charger versus dispensing unit, Since the smartphone 20 is mobile, in implementations it can observe patient patterns of when the medications are consumed or located at a patient's home or away from their home. The medication management companion induction charger may also include a “sim” that enables it to independently connect separately to one or more of the components of the medication adherence system.

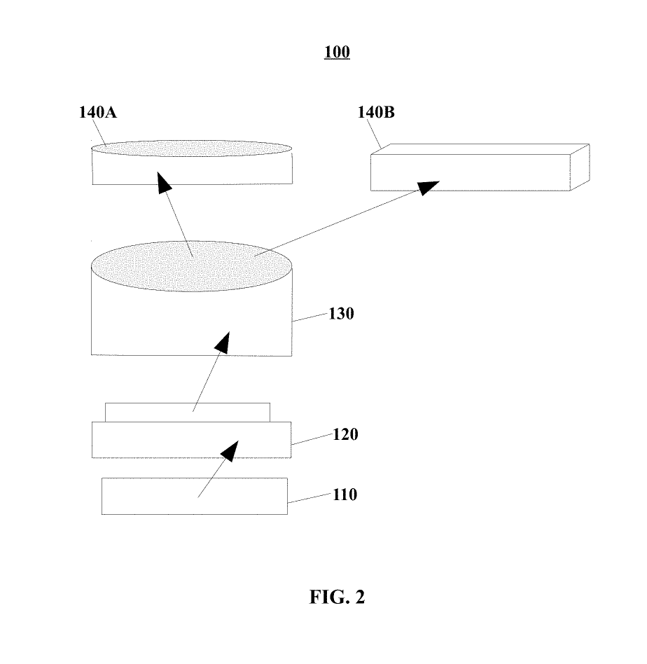

As shown in FIG. 2, a second embodiment of an exemplary medication adherence system 100 includes a smartphone 110, connector unit 120, dispensing unit 130 and a connected cap 140A or connected film layer 140B. In this embodiment, the connector unit 120 encloses the smartphone 110 and is linked directly to the connected cap 140A or the connected film layer 140B. The connected cap 140A or the connected film layer 140B include connectivity components and sensing components and battery instead of the dispensing unit 130. In one specific example, the connected cap 140A includes low battery Bluetooth® connectivity that can send a signal when the connected cap 140A has been turned, which indicates the patient is taking their medication. The connected cap 140A may change color as an indication to a patient it is time to take the medication. These types of settings are controlled through the smartphone 110 via the connector uni 120t.

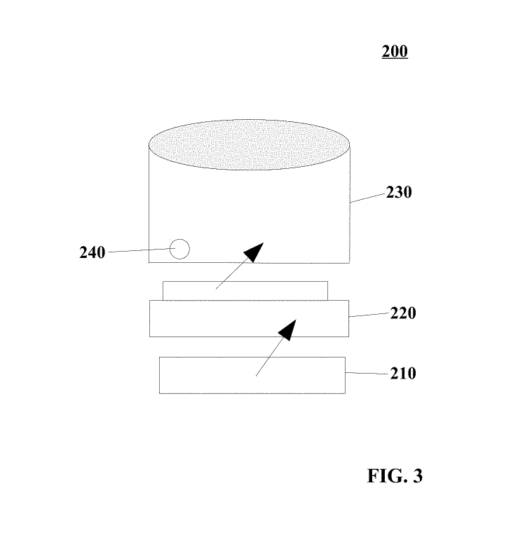

As shown in FIG. 3, a third embodiment of an exemplary medication adherence system 200 includes a smartphone 210, connector unit 220 and cream/lotion dispensing system 230. In this embodiment, the smartphone 210 and connector unit 220 can detect a moment in time when the cream/lotion dispensing system 230 changes configuration, as when it is pressed by the patient, causing the contents of the cream/lotion dispensing system 230 to flow through a portal 240. The changed configuration generates an airflow that is detected by the connector system 220, captured and recorded by the managing medicine adherence app 80 in the smartphone.

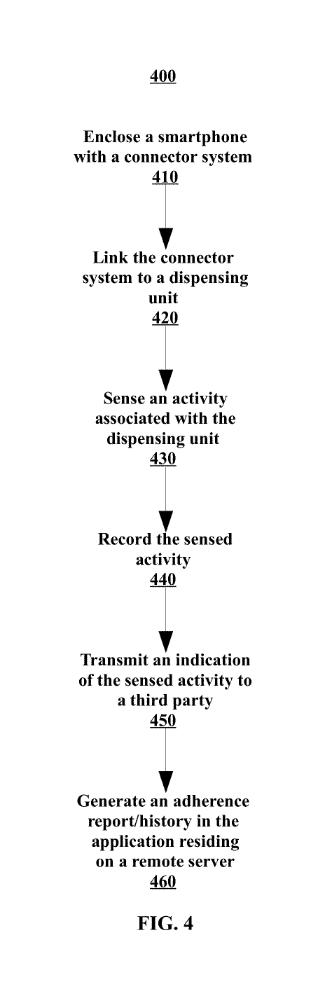

As shown in FIG. 4, a medication adherence process 400 includes enclosing (410) a smartphone with a connector system.

The process 400 links (420) the connector system to a dispensing unit. In embodiments, the dispensing unit may be a cream dispensing unit or a pill dispensing unit.

The process 400 senses (430) an activity associated with the dispensing unit. The activity may include any one or more of a movement, a temperature change, an air pressure change, and so forth.

The process 400 records (440) the sensed activity.

The process 400 transmits (450) an indication of the sensed activity to a third party. In embodiments, the third party can be an application residing on a remote server, a member of a medical team, a pharmacist, a family member, and so forth.

The process 400 optionally generates (460) an adherence report/history in the application residing on a remote server.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system comprising:
a smartphone linked to a dispensing unit by a connector system, the smartphone enabled to detect one or more items dispensed from the dispensing unit, the smartphone in communication with a content delivery network (CDN) including at least an adherence server.

2. The system of claim 1 wherein the dispensing unit is clasped to the connector system.

3. The system of claim 1 wherein the dispensing unit is integral to the connector system.

4. The system of claim 1 wherein the is selected from the group consisting of a cream dispensing unit and a pill dispensing unit.

5. A system comprising:
a smartphone, the smartphone comprising at least a processor, a memory and a display, the memory comprising at least an operating system and an adherence app;
a connector system, the connector system removably linking the smartphone to a medication dispensing container, the connector system comprising at least sensing technology that enables the connector system to read an activity from the medication dispensing container; and
a server in a network of interconnected computer devices, the server communicatively linked to the smartphone to enable interaction between the adherence app and an adherence process residing in the server.

6. The system of claim 5 wherein the at least sensing technology is selected from the group consisting of a device for the short-range wireless interconnection, Near Field Communication (NFC) and Radio Frequency Identification (RFID).

7. The system of claim 5 wherein the activity is selected from the group consisting of a movement, a temperature change, an air pressure change and a weight change.

8. The system of claim 5 wherein the adherence app record a user's behavior by the sensed activity.

9. The system of claim 8 wherein the sensed activity is transmitted to the adherence process in the server.

10. A medication adherence process comprises:
enclosing a smartphone with a connector system;
linking the connector system to a dispensing unit; and
sensing an activity associated with the dispensing unit.

11. The medication adherence process of claim 10 further comprising recording the sensed activity.

12. The medication adherence process of claim 11 further comprising transmitting an indication of the sensed activity to a third party.

13. The medication adherence process of claim 12 further comprising generates an adherence report/history in an application residing on a remote server from the indication.

14. The medication adherence process of claim 11 wherein the activity includes any one or more of a movement, a temperature change and an air pressure change.

15. The medication adherence process of claim 12 wherein the third party is a member of a medical team, a pharmacist or a family member.
